(19) United States
(12) Patent Application Publication
(10) Pub. No.: US 2009/0050146 A1
(43) Pub. Date: Feb. 26, 2009

(54) APPARATUS AND METHOD FOR INTUBATING AN AIRWAY OF A PATIENT

(75) Inventor: Michael Smith, Akron, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/191,722

(22) Filed: Aug. 14, 2008

Related U.S. Application Data
(60) Provisional application No. 60/957,467, filed on Aug. 23, 2007.

(51) Int. Cl. A61M 16/00

(52) U.S. Cl. 128/200.26

(57) ABSTRACT

An endotracheal tube has oppositely disposed proximal and distal ends fluidly connected by a ventilation lumen. An apparatus for intubating an airway of a patient with an endotracheal tube includes a housing and a motive mechanism supported by the housing and providing a user interface structure. A tube coupler is supported by the housing and is adapted for motion relative to the housing. The tube coupler is operatively coupled to the motive mechanism and is connectable to the proximal end of the endotracheal tube. A stylet coupler is supported by the housing and is adapted for motion relative to the housing. The stylet coupler is operatively coupled to the motive mechanism and is connectable to a proximal end of an intubation stylet. The motive mechanism is, operable to move the tube coupler in a first direction relative to the housing and to substantially simultaneously move the stylet coupler in a second direction relative to the housing in response to actuation of the user interface structure. A method of intubating an airway of a patient with an endotracheal tube is also described.

### RELATED APPLICATIONS

This application claims priority to the filing date of U.S. Provisional Application No. 60/957,467, filed Aug. 23, 2007, the subject matter of which is incorporated herein by reference.

### TECHNICAL FIELD

The present invention relates to an apparatus and method for airway management and, more particularly, to an apparatus and method for intubating an airway of a patient with an endotracheal tube.

### BACKGROUND OF THE INVENTION

Endotracheal intubation is a medical procedure in which an endotracheal tube is placed in the trachea of a patient to facilitate breathing or to permit the controlled introduction of certain gasses through the tube by an anesthesiologist or other medical personnel.

Trauma is a leading cause of death in the U.S. in patients between the ages of one and forty. The most common causes of trauma-related deaths are inadequate ventilation, inadequate circulation, or more massive hemorrhage for which there is little recourse. As critical care medicine developed over the last half-century, acute resuscitation techniques were established. Proper ventilation is important and may be very determinative of the outcome of a critical care event; if the patient cannot breathe, other critical care treatments may be provided in vain. However, it may be very difficult for a care provider to quickly and correctly intubate a patient in a high-stress situation, possibly with limited assistance. During the Vietnam War, for example, asphyxiation from upper airway obstruction or injury was a common cause of death in the field or en route to forward surgical facilities. Even in non-emergency situations, such as providing a patient with anesthesia, oxygen, or other gasses in a surgical setting, it is desirable that the intubation of the patient occur quickly and efficiently and without damage to the throat structures of the patient. Medical providers therefore must consider airway management, breathing, and circulatory problems in both critical care and routine procedures. Unfortunately, medical device development in this area has lagged behind the recognized need.

In, the most widely used protocol for intubating a patient (in both critical and routine applications), an endotracheal tube is inserted into the patient's airway with the assistance of a stiffening stylet. This stylet is generally a malleable metal rod placed within the lumen of the tube to reinforce the structure, of the, tube, which would otherwise bend under pressure, and thereby help advance the tube through the patient's mouth and throat. Routinely, a laryngoscope is used to visualize the patient's airway and allow the user to directly observe the passage of the endotracheal tube and enclosed stylet, particularly in the initial stages of the procedure.

The stylet is used to stiffen the tube for passage through the patient's mouth and into the throat. However, it may be undesirable for the stylet to remain inside the tube past the vocal cords due to the potential for injury to delicate airway structures. Therefore, as the user is advancing the tube in the vicinity of the vocal cords with one (usually right) hand, normally with the assistance of a laryngoscope in the other (usually lefty hand, an assistant is needed to at least partially withdraw the stylet from the tube. Once the user determines that the tube has been placed as desired, the stylet is completely removed from the tube (if not previously done) and ventilation of the patient commences.

This intubation process is resource-intensive and may be prone to errors. Particularly in a critical-care situation, the assistant is prevented from performing other, possibly time-sensitive, tasks while helping the user with the intubation. The presence of two operators (the user and the assistant) could adversely affect the steadiness of the tube and/or stylet during the procedure, which may already be precarious if the intubation is being performed in a moving vehicle. Miscommunication could occur between the user and assistant, which could result in the stylet being withdrawn at a different speed and/or time than desired. The assistant could accidentally drag the tube out of position through the friction of the withdrawing stylet. Because of the delicate nature of the intubation procedure, any of these or other complications could substantially increase the time needed to perform the procedure and/or the risk of injury to the patient.

Even if the user sets aside the laryngoscope and grasps the stylet with the non-dominant hand, thus obviating the need for an assistant, it can be very difficult for a single person to simultaneously withdraw the stylet and to either hold the tube in position or continue advancing the tube, while accomplishing these tasks in a smooth and controlled manner.

### SUMMARY OF THE INVENTION

In an embodiment of the present invention, an apparatus for intubating an airway of a patient with an endotracheal tube is described. The endotracheal tube has oppositely disposed proximal and distal ends fluidly connected by a ventilation lumen. The apparatus includes a housing and a motive mechanism supported by the housing and providing a user interface structure. A tube coupler is supported by the housing and is adapted for motion relative to the housing. The tube coupler is operatively coupled to the motive mechanism and is connectable to the proximal end of the endotracheal tube. A stylet coupler is supported by the housing and is adapted for motion relative to the housing. The stylet coupler is operatively coupled to the motive mechanism and is connectable to a proximal end of an intubation stylet. The motive mechanism is operable to move the tube coupler in a first direction relative to the housing and to substantially simultaneously move the stylet coupler in a second direction relative to the housing in response to actuation of the user interface structure.

In an embodiment of the present invention, an apparatus for intubating an airway of a patient with an endotracheal tube is described. The endotracheal tube has oppositely disposed proximal and distal ends fluidly connected by a ventilation lumen. The apparatus includes a housing and a motive mechanism supported by the housing and providing, a user interface structure. A tube coupler is supported by the housing and is adapted for motion relative to the housing. The tube coupler is operatively coupled to the motive mechanism and is connectable to the proximal end of the endotracheal tube. A stylet coupler is supported by the housing and is adapted for motion relative to the housing. The stylet coupler is operatively coupled to the motive mechanism and is connectable to a proximal end of an intubation stylet. The motive mechanism is operable to move the tube coupler in a first direction relative to the housing and to move the stylet coupler in a second direction relative to the housing in response to actuation of the user interface structure. The first direction is substantially opposite the first direction.

In an embodiment of the present invention, a method of intubating an airway of a patient with an endotracheal tube is described. The endotracheal tube has oppositely disposed proximal and distal ends fluidly connected by a ventilation lumen. An intubation apparatus is provided, the intubation apparatus having a housing, a motive mechanism supported by the housing and providing a user interface structure, a tube coupler operatively coupled to the motive mechanism, and a stylet coupler operatively coupled to the motive mechanism. A stylet is inserted into the ventilation lumen. The stylet is connected to the stylet coupler. The endotracheal tube is connected to the tube coupler. The endotracheal tube and stylet are inserted into the airway of the patient. The user interface structure is selectively actuated. The tube coupler is moved in a first direction relative to the housing substantially simultaneously with moving the stylet coupler in a second direction relative to the housing, responsive to actuation of the user interface structure.

### BRIEF DESCRIPTION OF THE DRAWINGS

For a better understanding of the invention, reference may be made to the accompanying drawings, in which:

FIG. 1 is a side view of a first embodiment of the present invention in a use environment;

FIG. 2 is a partial side view of the embodiment of FIG. 1;

FIG. 3A is a schematic side view of the embodiment of FIG. 1 in an initial condition;

FIG. 3B is a schematic side view-of the embodiment of FIG. 1 in an actuated condition;

FIG. 4A is a schematic side view of a second embodiment of the present invention in an initial condition;

FIG. 4B is a schematic side view of the embodiment of FIG. 4A in an actuated condition; and

FIG. 4C is a cross-sectional view taken along the line 4C-4C in FIG. 4A.

### DESCRIPTION OF EMBODIMENTS

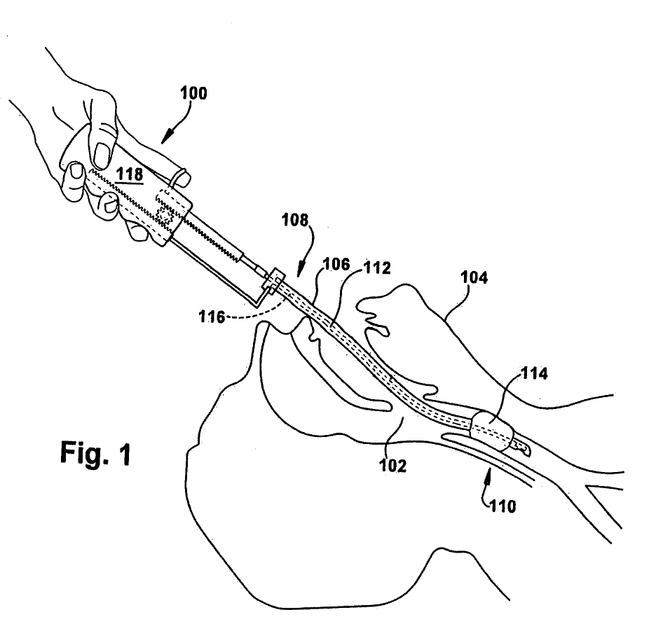

In accordance with the present invention, FIG. 1 depicts an apparatus 100 for intubating an airway 102 of a patient 104 with an endotracheal tube 106. The endotracheal tube 106 has oppositely disposed proximal and distal tube ends 108 and 110, respectively, fluidly connected by a ventilation lumen 112. An anchoring structure 114 may be provided at or near the distal tube end 110 to assist in maintaining the endotracheal tube 106 in the desired position once intubation is complete. The endotracheal tube 106 for use with the present invention may be a standard item or may include one or more specialized features for use with the apparatus 100. Like all structures described herein, the endotracheal tube 106 could be made of any material, and in any manner, as desired for a particular application of the present invention.

As depicted in FIG. 1, an intubation stylet 116 may be located at least partially within the ventilation lumen 112, to help reinforce and shape the endotracheal tube 106 for insertion into the airway 102. The stylet 116 may be a standard item or may include one or more specialized features or structures for use with the apparatus 100. For example, the stylet 116 could be of the conventional malleable metal type. Additionally or alternatively, the stylet 116 could include a light or a remote viewing device, such as a fiber optic camera, to facilitate visualization of the stylet or endotracheal tube 106 during intubation.

The arrangement of FIG. 1 can be considered to show an initial or “primary intubation” configuration, in which the stylet/tube combination initially enters the airway 102 and is directed toward the vocal cords (omitted from FIG. 1 for clarity, but located generally in the area in which the distal tube end 110 is shown). In this initial configuration, the stylet 116 may be considered fully inserted into the endotracheal tube 106 for use, despite the extension of some portion of the stylet 116 from the proximal tube end 108. Though not depicted here, a portion of the stylet 116 may also or instead extend from the distal tube end 110 in the initial configuration.

FIG. 1 provides an exterior view of the apparatus 100 which may be supplied integrally, in combination with, or separately from the endotracheal tube 106 and/or the stylet 116. For example, a reusable apparatus 100 could be packaged in combination with a plurality of single-use stylets 116 and/or endotracheal tubes 106. Whether sold separately or in a combination package, at least one of the stylet 116 and the endotracheal tube 106 may be specifically adapted for engagement with the apparatus 100. At least one of the stylet 116 and the endotracheal tube 106 may also or instead be a commercially available item. In addition, one of ordinary skill in the art could readily provide an adapter (not shown) to enable any suitable type of stylet 116 and/or endotracheal tube 106 to be used with the apparatus 100. The specific types of stylet 116 and endotracheal tube 106 which may be used with the apparatus 100 are not limited by the present invention.

As shown in the exterior view, the apparatus 100 includes a housing 118. The housing 118 may serve to enclose, support, and/or protect other structures of the apparatus 100. The material and/or configuration of the housing 118 may be chosen to provide desired properties, such as ease of sterilization. The housing 118 may be economically shaped to at least partially mate with a hand of a user (not shown) and may include one or more cushioned and/or textured areas for ease and comfort in gripping, retention, and/or manipulation. The configuration of the housing 118 may be chosen in response to the shapes of one or more structures to be housed within the housing.

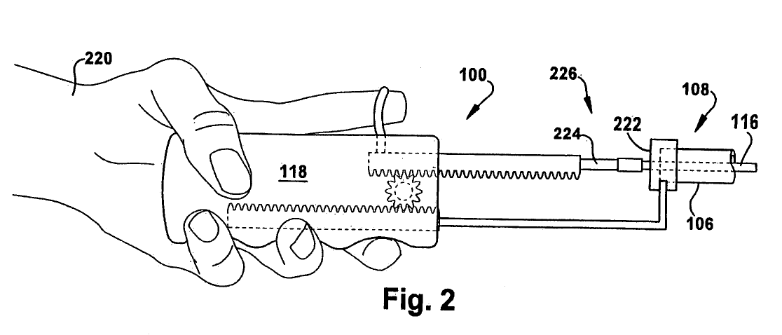

FIG. 2 depicts the, apparatus 100 in a hand 220 of a user. The apparatus 100, or components thereof, may be provided in a range of sizes, shapes, and/or configurations for use with different stylets, endotracheal tubes, and/or user hand sizes/shapes. For example, when the housing 118 is ergonomically shaped, left-handed and right-handed versions may be provided. The user may hold the housing 118 in the overhand or “pistol grip” manner shown in FIG. 2, even if the housing 118 is not ergonomically shaped.

A tube coupler 220 is supported by the housing 118 and is adapted for motion relative to the housing, as will be discussed below. The tube coupler 220 is connectable to the proximal tube end 108, and such connection may result from integral formation or otherwise permanent attachment of the tube coupler 220 to at least a portion of the endotracheal tube 106.

A stylet coupler 224 is supported by the housing 118 and is adapted for motion relative to the housing, as will be discussed below. The stylet coupler 224 is connectable to a proximal stylet end 226, and such connection may result from integral formation or otherwise permanent attachment of the stylet coupler 224 to at least a portion of the stylet 116.

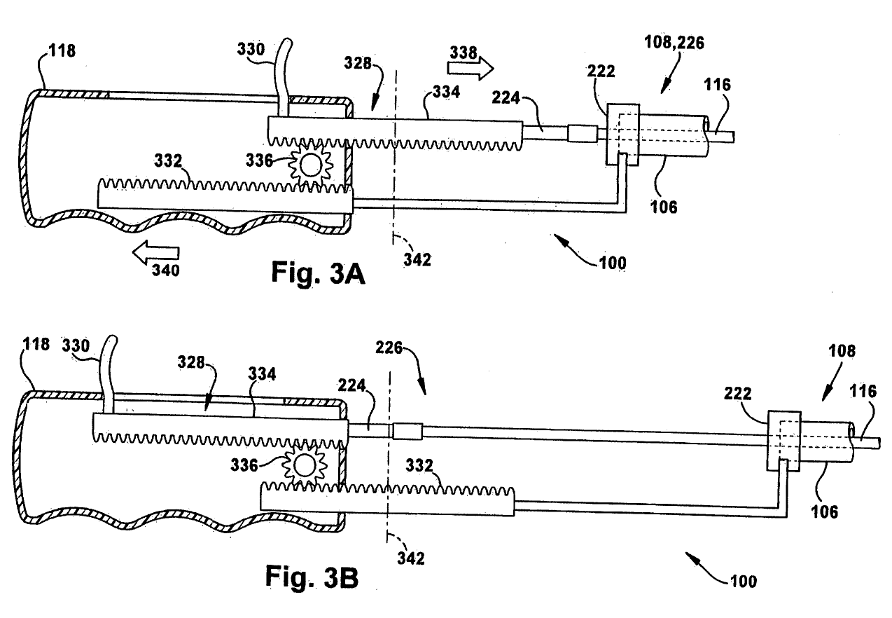

FIGS. 3A and 3B schematically depict a first embodiment of the apparatus 100 in initial and actuated conditions, respectively. The housing 118 is omitted from these views to facilitate depiction of the inner workings of the apparatus 100, such as the motive mechanism 328 shown.

The motive mechanism 328 is supported by the housing 118 and may be of any suitable type. The motive mechanism 328 provides a user interface structure 330 of any suitable type, such as the depicted “trigger” structure. The motive mechanism 328 moves the tube coupler 222 and the stylet coupler 224 in response to actuation of the user interface structure 330. The user interface structure 330 may be any structure which a user can manipulate, manually and/or automatically, to actuate the apparatus 100. For example, the user interface structure could be a foot pedal, push button, remote control, squeeze bulb, slider, wheel, lever, touch sensor, electric or electromechanical switch, or the like, or any combination thereof.

In the first embodiment, the motive mechanism is of a rack-and-pinion type, with a tube rack 332 and a stylet rack 334 operatively connected by a pinion gear 336. The tube rack 332 may be operatively coupled to the endotracheal tube 106, possibly through a connecting member such as the tube coupler 222. The stylet rack 334 may be operatively coupled to the stylet 116, possibly through a connecting member such as the stylet coupler 224.

Regardless of how the tube coupler 222 and stylet coupler 224 are operatively coupled to the motive mechanism 328, the motive mechanism may be operable to move the tube coupler in a first direction 338 relative to the housing 118 and to move the stylet coupler in a second direction 340 relative to the housing, as shown in the actuated condition of FIG. 3B. (The housing 118 is considered to stay relatively stationary, as indicated by the reference axis 342 in FIGS. 3A and 3B.) In other words, the user interface structure 330 may transmit force from a user to at least one of the tube coupler 222 and the stylet coupler 224 through the motive mechanism 328.

In the motive mechanism 328 depicted in FIGS. 3A and 3B, for example, the user interface structure 330 is substantially rigidly connected to the stylet rack 334, as is the stylet 116. When the user slides the user interface structure 330 in the first direction 338 relative to the housing 118, the stylet rack 334 is also pulled in the first direction by the actuation force exerted by the user. Through linkage with the pinion gear 336, a portion of the actuation force may be redirected to move the tube rack 332 in the second direction 340.

When the apparatus 100 is operatively connected to an endotracheal tube 106 and a stylet 116 and actuated via the user interface structure 330, the motions of the tube coupler 222 and the stylet coupler 224 act to advance the endotracheal tube while withdrawing the stylet. The tube coupler 222 could be moved in the first direction 338 substantially simultaneously with movement of the stylet coupler 224 in the second direction 340. The first direction 338 could be substantially parallel to the second direction 340, and could also or instead be substantially opposite the second direction 340. The motive mechanism 328 may be used to move the tube coupler 222 and/or the stylet coupler 224 (and by extension the endotracheal tube 106 and/or the stylet 116) in any desired direction, sequence, or manner suitable to provide the desired retraction of the stylet from the endotracheal tube as the endotracheal tube is either maintained in position or advanced into the airway 102 of the patient 104.

The motive mechanism 328 may include a mechanical advantage device (not shown), which could act to enhance and/or reduce the effect of the force exerted by the user upon certain structures of the apparatus 100. For example, a gear train could be provided to the first embodiment, rather than the depicted pinion gear 336, and thereby provide a differential relationship between the movements of the user interface structure 330, the tube coupler 222, and/or the stylet coupler 224. The mechanical advantage device, when preset, could use mechanical power, electrical power, electromechanical power, hydraulic power, pneumatic power, or the like, or any combination thereof, to provide desired ratios of motion and/or force between two or more of the user interface structure 330, the endotracheal tube 106, and the stylet 116. A suitable manual or automatic mechanical advantage device could be readily designed by one of ordinary skill in the art for a particular application of the present invention.

It is contemplated that motion of the tube coupler 222 in the first direction 338 could at least partially disconnect the endotracheal tube 106 from the apparatus 100. For example, an ejector (not shown) could extend from the housing 118, and motion of the tube coupler 222 past the ejector could disengage the endotracheal tube 106 from the tube coupler 222. Such an ejector could facilitate complete separation of the apparatus 100 and stylet 116 from the endotracheal tube 106 while preserving one-handed use of the apparatus as desired.

One or more intubation accessories (not shown) could be provided to the apparatus 100, either integrally or in a separate or separable manner. For example, a suction device, an illumination device, a remote viewing device (e.g., a fiber optic lens), a fluid supply device, a measuring device, and/or a remote access device (e.g., an endoscopic tool) could be provided. Any such intubation accessory could act at one or both of the proximal and distal tube ends 108 and 110, possibly with the assistance of the endotracheal tube 106, and could readily be provided by one of ordinary skill in the art.

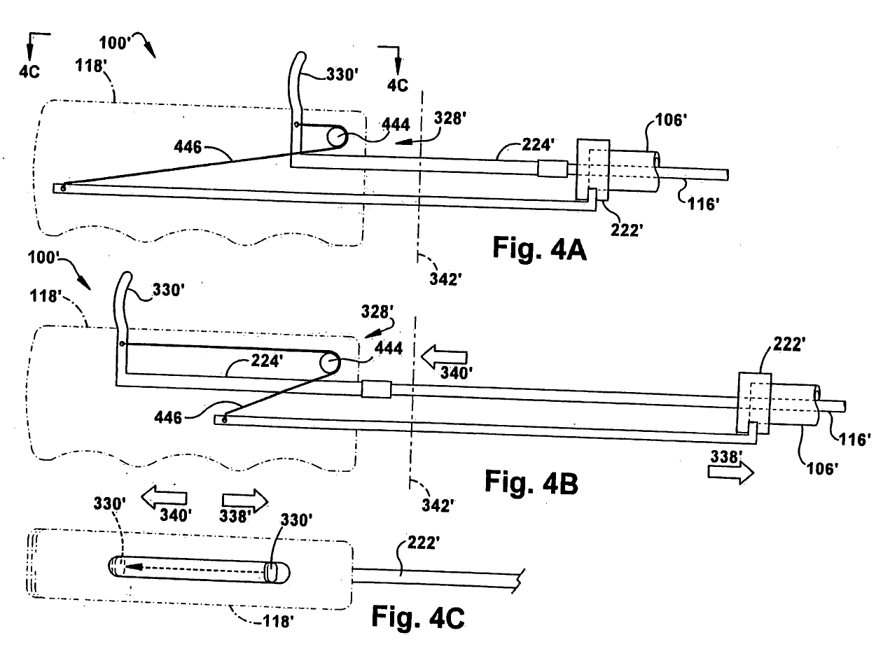

FIGS. 4A, 4B, and 4C depict an apparatus 100′ in accordance with a second embodiment of the present invention. Features of FIGS. 4A, 4B, and 4C that are the same as or similar to those described with regard to FIGS. 1-3B are given the same reference numbers with the addition of a single prime. Description of common elements and operation similar to those in the first embodiment will not be repeated with respect to the second embodiment.

The second embodiment of the apparatus 100′ differs from the first embodiment primarily in the structure of the motive mechanism 328′. As can be seen in FIG. 4A, a peg 444 is affixed to the housing 118′ (shown in phantom line in FIGS. 4A, 4B, and 4C), or otherwise held stationary with respect to at least one of the tube coupler 222′ and the stylet coupler 224′. The peg 444 may be rotatable or may instead be a fixed knob or protrusion. A flexible strand 446, which could be a wire, cable, string, chain, fiber, or other elongate, flexible material, is affixed to both the tube coupler 222′ and the stylet coupler 224′ and is in slidable contact with the peg 444. As the motive mechanism 328′ is manipulated through the user interface structure 330′, the stylet coupler 224′ is moved in the second direction 340′, from the initial condition of FIG. 4A to the actuated condition of FIG. 4B. The moving stylet coupler 224′ pulls the strand 446 slides or tracks against the peg 444, and the strand 446 moves the tube coupler 222′ in the first direction 338′. In this manner, the motive mechanism 328′ moves the tube coupler 222′ in a first direction 338′ relative to the housing 118′ and moves the stylet coupler 224′ in a second direction 340 relative to the housing 118′.

In the second embodiment of the apparatus, the user interface structure 330′ appears to be formed integrally with the stylet coupler 224′ and the stylet 116′. Whether or not these structures are formed in a unitary manner, a “connection” (as referenced herein) still exists between the stylet coupler 224′ and the stylet 116′. However, it may be desirable in some applications of the present invention to design the tube coupler 222′ and/or stylet coupler 224′ for a detachable connection with the endotracheal tube 106′ and/or stylet 116′, respectively, to facilitate reuse of the apparatus 100′ with a series of disposable endotracheal tubes and/or stylets.

FIG. 4C is a cross-sectional view taken along line 4C-4C of FIG. 4A and depicts one possible relationship of the user interface structure 330′ with the housing 118′. (The endotracheal tube 106′ and stylet 116′ have been omitted in FIG. 4C for clarity.) In FIG. 4C, the user interface structure 330′ is shown as a small-diameter trigger-type structure which is constrained in, and guided by, a slot 448 for one-dimensional movement in the first and second directions 338′ and 340′ with respect to the housing 118′. The dimensions of the slot 448 may be chosen in response to a size or shape of the user interface structure 330′. For example, if the user interface structure 330′ is a flat slider-type structure which is wide enough to accommodate a user's fingertip, the slot 448 may need to be wider than that shown in FIG. 4C. The user interface structure 330′ and tube coupler 222′ can be selectively moved between the initial condition (solid line) and actuated condition (phantom line) during manipulation of the apparatus 100′ by the user.

In order to use any embodiment of the apparatus 100 to intubate the airway 102 of a patient 104, a stylet 116 is at least partially inserted into the ventilation lumen 112 of the endotracheal tube 106. The stylet 116 is connected to the stylet coupler 224. In many applications of the present invention, the stylet 116 will be inserted into the endotracheal tube 106 before being connected to the stylet coupler 224. However, the structure of a specific embodiment of the apparatus 100 may make any of the described steps desirable to perform in different sequences in different applications of the present invention.

The endotracheal tube 106 is connected to the tube coupler 222, and the endotracheal tube 106 (with stylet 116 inserted) can be inserted into the airway 102 of the patient 104. The stylet 116 will stiffen and help guide the endotracheal tube 106 within the airway 102. The apparatus 100 may be manipulated by a single user to intubate the patient 104, with the user optionally using just a single hand to perform the intubation due to the structural features provided in certain embodiments of the apparatus 100. Any suitable intubation accessories (not shown) may be provided to the apparatus 100 or separately for use concurrently with the apparatus 100 to facilitate intubation.

Once the endotracheal tube 106, containing the stylet 116 has reached a desired level of insertion the user selectively actuates the user interface structure 330. In response to the actuation of the user interface structure 330 the tube coupler 222 moves in the first direction 338 relative to the housing 118 and the stylet coupler 224 moves in the second direction 340 relative to the housing 118. As a result of these relative movements, the stylet 106 is at least partially withdrawn from the endotracheal tube 116.

The endotracheal tube 116 is completely disengaged from the apparatus 100, and the stylet 106 completely withdrawn, to place the endotracheal tube 116 into an available condition for ventilating the patient 104. Those portions of the apparatus 100, endotracheal tube 106, and/or stylet 116 which are reusable may be sterilized or otherwise prepared and/or stored for reuse, and the disposable portions may be discarded.

While aspects of the present invention have been particularly shown and described with reference to the preferred embodiment above, it will be understood by those of ordinary skill in the art that various additional embodiments may be contemplated without departing from the spirit and scope of the present invention. For example though the tube rack 332, endotracheal tube 106, and tube coupler 222 are described herein as being separate structures, one or more of these structures could be integrally formed or provided, and these structures need not be clearly delineated from one another. Similarly, the stylet rack 334, stylet 116, and/or the stylet coupler 224 could be integrally formed or provided, and may likewise be difficult to differentiate. The mechanical advantage device, when present, could include at least one gear train, block and tackle, piston, stepper motor, spring, and/or any other suitable force multiplying or dividing device or combination thereof. Additional structures could be present in the apparatus 100 to assist in guiding, anchoring, steadying, connecting, or otherwise manipulating the described structures as desired. The described structures could include slots, bends, or any other features operative to prevent physical interference with other structures of the apparatus 100. Any suitable motive mechanism could be used, as long as the chosen structure is operative to move the tube coupler 222 and stylet coupler 224 as described. The initial conditions and actuated conditions depicted do not limit the possible arrangements in which the apparatus 100 may be placed; there are a plurality of intermediate arrangements between and/or beyond the initial and actuated conditions shown in which the apparatus 100 may be placed or used. The patient could be human or could be a non-human animal, with appropriate structural modifications to the apparatus 100 as needed. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

Having described the invention, I claim:

1. An apparatus for intubating an airway of a patient with an endotracheal tube, the endotracheal tube having oppositely disposed proximal and distal ends fluidly connected by a ventilation lumen, the apparatus comprising:
a housing;
a motive mechanism supported by the housing and providing a user interface structure;
a tube coupler supported by the housing and adapted for motion relative to the housing, the tube coupler being operatively coupled to the motive mechanism and connectable to the proximal end of the endotracheal tube; and
a stylet coupler supported by the housing and adapted for motion relative to the housing, the stylet coupler being operatively coupled to the motive mechanism and connectable to a proximal end of an intubation stylet;
the motive mechanism being operable to move the tube coupler in a first direction relative to the housing and to substantially simultaneously move the stylet coupler in a second direction relative to the housing in response to actuation of the user interface structure.

2. The apparatus of claim 1, wherein the first direction is substantially parallel to the second direction.

3. The apparatus of claim 1, wherein the first direction is substantially opposite the second direction.

4. The apparatus of claim 1, wherein at least one of the intubation stylet and the endotracheal tube is adapted for engagement with the apparatus.

5. The apparatus of claim 1, wherein the housing is ergonomically shaped to at least partially mate with a hand of a user.

6. The apparatus of claim 1, wherein the user interface structure transmits force from a user to at least one of the tube coupler and the stylet coupler through the motive mechanism, and the motive mechanism includes a mechanical advantage device.

7. The apparatus of claim 1, wherein motion of the tube coupler in the first direction at least partially disconnects the endotracheal tube from the apparatus.

8. The apparatus of claim 4, including at least one intubation accessory selected from the group comprising: a suction device, an illumination device, a remote viewing device, a fluid supply device, a measuring device, and a remote access device.

9. An apparatus for intubating an airway of a patient with an endotracheal tube, the endotracheal tube having oppositely disposed proximal and distal ends fluidly connected by a ventilation lumen, the apparatus comprising:
a housing;
a motive mechanism supported by the housing and providing a user interface structure;
a tube coupler supported by the housing and adapted for motion relative to the housing the tube coupler being, operatively coupled to the motive mechanism and connectable to the proximal end of the endotracheal tube; and
a stylet coupler supported by the housing and adapted for motion relative to the housing, the stylet coupler being operatively coupled to the motive mechanism and connectable to a proximal end of an intubation stylet;
the motive mechanism being operable to move the tube coupler in a first direction relative to the housing and to move the stylet coupler in a second direction relative to the housing in response to actuation of the user interface structure, the first direction being substantially opposite the first direction.

10. The apparatus of claim 9, wherein the motive mechanism moves the tube coupler in the first direction relative to the housing substantially simultaneously with moving the stylet coupler in the second direction relative to the housing.

11. The apparatus of claim 9, wherein the first direction is substantially parallel to the second direction.

12. The apparatus of claim 9, wherein at least one of the intubation stylet and the endotracheal tube is adapted for engagement with the apparatus.

13. The apparatus of claim 9, wherein the housing is ergonomically shaped to at least partially mate with a hand of a user.

14. The apparatus of claim 9, wherein the user interface structure transmits force from a user to at least one of the tube coupler and the stylet coupler through the motive mechanism, and the motive mechanism includes a mechanical advantage device.

15. The apparatus of claim 9, wherein motion of the tube coupler in the first direction at least partially disconnects the endotracheal tube from the apparatus.

16. The apparatus of claim 9, including at least one intubation accessory selected from the group comprising: a suction device, an illumination device, a remote viewing device, a fluid supply device, a measuring device, and a remote access device.

17. A method of intubating an airway of a patient with an endotracheal tube, the endotracheal tube having oppositely disposed proximal and distal ends fluidly connected by a ventilation lumen, the method comprising the steps of:
providing an intubation apparatus having a housing, a motive mechanism supported by the housing and providing a user interface structure, a tube coupler operatively coupled to the motive mechanism, and a stylet coupler operatively coupled to the motive mechanism;
inserting an intubation stylet into the ventilation lumen;
connecting the intubation stylet to the stylet coupler;
connecting the endotracheal tube to the tube coupler;
inserting the endotracheal tube and intubation stylet into the airway of the patient;
selectively actuating the user interface structure; and
moving the tube coupler in a first direction relative to the housing substantially simultaneously with moving the stylet coupler in a second direction relative to the housing, responsive to actuation of the user interface structure.

18. The method of claim 17, wherein the first direction is substantially parallel to the second direction.

19. The method of claim 17, wherein the first direction is substantially opposite the second direction.

20. The method of claim 17, wherein the step of moving the tube coupler in a first direction relative to the housing substantially simultaneously with moving the stylet coupler in a second direction relative to the housing, responsive to actuation of the user interface structure includes the steps of:
transmitting force from a user to at least one of the tube coupler and the stylet coupler through the motive mechanism; and
at least one of enhancing and reducing the user force during transmission through a mechanical advantage device;

21. The method of claim 17, including the step of at least partially disconnecting the endotracheal tube from the apparatus via motion of the tube coupler in the first direction.

22. The method of claim 17, including the step of providing at least one intubation accessory selected from the group comprising: a suction device, an illumination device, a remote viewing device, a fluid supply device, a measuring device, and a remote access device.

23. The method of claim 17, wherein the steps of selectively actuating the user interface structure and moving the tube coupler in a first direction relative to the housing substantially simultaneously with moving the stylet coupler in a second direction relative to the housing, responsive to actuation of the user interface structure are performed responsive to actions of a single user.
